(19) United States
(12) Patent Application Publication
(10) Pub. No.: US 2015/0160221 A1
(43) Pub. Date: Jun. 11, 2015

(54) BIOMARKERS FOR BREAST CANCER PREDICTIONS AND DIAGNOSES

(71) Applicants: Tao Wang, Suzhou (CN); Xiangyun Qu, Suzhou (CN); Fei Chen, Suzhou (CN)

(72) Inventors: Tao Wang, Suzhou (CN); Xiangyun Qu, Suzhou (CN); Fei Chen, Suzhou (CN)

(73) Assignee: SUZHOU MICRODIAG BIOMEDICINE CO., LTD, Suzhou, Jiangsu (CN)

(21) Appl. No.: 14/371,099

(22) PCT Filed: Jan. 9, 2012
(86) PCT No.: PCT/CN2012/070151

§ 371 Date: Dec. 12, 2014

(51) Int. Cl. G01N 33/574; C12Q 1/68

(52) U.S. Cl. CPC G01N 33/57415; C12Q 1/6886; C12Q 2600/158; G01N 2333/8139; C12Q 2600/112

(57) ABSTRACT

The applications of CST4 gene, mRNA of CST4, cDNA of the splice variants of CST4, the amplicons of the CST4 primers, cystatin S protein coded by CST4 and epitope peptide of cystatin S in the predictions and diagnoses of human breast cancers, is disclosed in this invention. It can be used for the diagnosis and real time monitoring of breast cancers and tumor prognosis predictions. This methods disclosed are verified with large-scale clinical trials, and are of significant reliability and sensitivity.

### TECHNOLOGY SCOPE

The present invention is of biomedical technology. Biomarkers for breast cancer and their applications for breast cancer diagnosis, dynamic detection and progression determination are mentioned in this invention. Test reagents and kits, and their respective protocols are also claimed in this invention.

### BACKGROUND

According to a report on cancer by WHO, around 1.2 million cases of breast cancer occurrence are reported annually. Over 400 thousand breast cancer cases are reported annually in China, and the incidence is increasing. In metropolitan areas such as Beijing and Shanghai, breast cancer incidence is higher than any other cancers. In incidence of breast cancer in Shanghai increased from 17.7/100000 to 70/100000 in last thirty years. The rank of incidence of breast cancer among all malignant tumors increased from No. 2 to No. 1. The 5-year overall survival rate is less than 60%.

Although the early detections and 5-year survival rate improved significantly recently, 540 thousand deaths due to breast cancer was reported in World Cancer Report 2008 (by WHO). The domestic mortality rate of breast cancer increases by 3% annually. Many problems need solutions in the prevention and treatment of breast cancer, including early detections and interventions of breast cancers, breast cancer treatment evaluation, tumor prognosis monitoring and accurate predictions for breast cancer recurrence and metastasis for post-treatment patients.

Diagnosis methods for breast cancer with high sensitivity and specificity is critical for early screening of breast cancers and for improving the prognosis of the patients. Cystatin superfamily is a series of proteins with cathepsins inhibition activity. They play important roles in the occurrence and development of cancers. Proteins belonging to the cystatin superfamily reversibly bind cystein protease in tissues and body fluids, to avoid the overactivity of cathepsins. Cystatin C is the ligand for cathepsin B with the highest affinity. It has increased expression levels in tumors of ovarian cancers and head and neck cancers. Stefin A (a member of cystatin superfamily) has increased expression in non-small cell lung cancer (NSCLC) tumors. Expression of mRNA of stefin B is inhibited in meningiomas. Cystatin F (or leukocystatin, CMAP) has high expression in various tumors. Investigations show that the increased cystatins expression is possibly due to the participation of cathepsin in the occurrence and prolification of tumors, during which cathepsin expression rises, leading to the increase of the expression of cystatin to inhibit the overactivity of cathepsin through a feedback mechanism. However, it's noted however that the expression of cystatin is not always correlated with tumor growth positively. For instance, low expression of cystatin C indicates late stage, poor prognosis and high metastasis possibility among glioma patients. Both mRNA and protein expression were investigated and the conclusion was verified in both studies.

In this invention, the strong correlation of CST4 (a member of cystatin superfamily) and its splices with breast cancer tumors is verified. CST4, or cystatin S, is one of cystein protease inhibitors, with 141 amino acid residues. Cystatin S is found in various body fluids and secretions such as tears, saliva, plasma and serum.

### SUMMARY OF THE INVENTION

One of the objectives of this invention provides noval applications of CST4 gene, mRNA of CST4, cDNA of splices of CST4, amplicons for CST4-specific primers, cystatin S protein coded by CST4 gene and epitope peptide of cystatin S. These applications are of significant importance in developing new diagnosis methodologies for breast cancers.

Technologies Included in this Invention are Described as Following:

1. Applications of CST4 gene, mRNA of CST4, cDNA of splices of CST4, amplicons for CST4-specific primers, cystatin S protein coded by CST4 gene and epitope peptide of cystatin S in the diagnosis and prediction of breast cancers. Sequence of CST4 gene is presented in SEQ ID No.42. The probe for CST4 gene, mRNA of CST4 and cDNA of splices of CST4 has sequence as shown in SEQ ID No. 3. Specific primers of the amplicon have sequences shown in SEQ ID No.1, 4, 6, 8, 10, 12, 14, 16, 18, 20 (primer 1) and in SEQ ID No.2, 5, 7, 9, 11, 13, 15, 17, 19, 21 (primer 2). Sequence in SEQ ID No.1 pairs with sequence in SEQ ID No.2. Sequence in SEQ ID No.4 pairs with sequence in SEQ ID No.5. Sequence in SEQ ID No.6 pairs with sequence in SEQ ID No.7. Sequence in SEQ ID No.8 pairs with sequence in SEQ ID No.9. Sequence in SEQ ID No.10 pairs with sequence in SEQ ID No.11. Sequence in SEQ ID No.12 pairs with sequence in SEQ ID No.13. Sequence in SEQ ID No.14 pairs with sequence in SEQ ID No.15. Sequence in SEQ ID No.16 pairs with sequence in SEQ ID No.17. Sequence in SEQ ID No.18 pairs with sequence in SEQ ID No.19. Sequence in SEQ ID No.20 pairs with sequence in SEQ ID No.21. Sequence the epitope peptide of cystatin S is shown in SEQ ID No.50. Diagnosis and screening herein mean metastasis and micro-metastasis of breast cancers, pTNM staging, real time monitoring of the tumor during cancer treatment and prognosis predictions. It should be noted that these sequences should not limit the scope of this invention. All sequences of their functions are included in this invention.

The second objective of this invention is to provide several capturers, which specifically interact with breast cancer markers.

In order to realize the above purposes, the technologies are described as following:

Capturers for breast cancer markers, wherein the capturers are capturers for breast cancer markers for breast cancer prediction and diagnosis. The breast cancer markers are CST4 gen, mRNA of CST4, cDNA of splices of CST4, amplicons for CST4-specific primers, cystatin S protein coded by CST4 gene and epitope peptide of cystatin S.

Sequences of the primers mentioned in 3) are presented in SEQ ID No.1-2.

The sequence of the probe mentioned in 2) is presented in SEQ ID No.3.

Sequence of the amplicon mentioned in 3) is presented in SEQ ID No.43.

Capturers mentioned herein are those molecules that specifically recognize cystatin S or its epitope peptide.

The sequence of epitope peptide of cystatin S is shown in SEQ ID No.50.

The third objective of this invention is to provide novel applications for capturers. Testing kits and their respective protocols based on the capturers. As novel methodologies, these applications and testing kits for breast cancer detections feature high accuracy, easy operation and feasibility for large-scale clinical practice.

In order for the realization of the mentioned objective, the technologies are described as following.

Applications of the capturers in the manufacturing of testing reagents and kits for breast cancer detections.

All diagnostic kits with these capturers.

Details of the diagnostic kits are as following.

1) Real time and quantitative testing kits for mRNA of CST4 using TaqMan probes. The primers sequences are shown in SEQ ID No. 1-2. The sequence of the probe is shown in SEQ ID No.3.

2) Real time and quantitative testing kits for mRNA of CST4 using fluorescent dyes as probes. The primers sequences are shown in SEQ ID No. 1-2. The sequences of the primers for internal reference are shown in SEQ ID No.30-31. Or

3) Quantitative testing kits for mRNA of CST4 based on nucleic acid based amplification (NASBA) or transcription-median amplification (TMA). Both kits include primers and probes for CST4, whose sequences are shown in SEQ ID No.2, 32 (for primers) and 3 (for probe). Or

4) Quantitative testing kits for mRNA of CST4 based on ligase chain reaction (LCR). Four probes are included whose sequences are shown in SEQ ID No.33-36. Or

5) Quantitative testing kits for mRNA of CST4 based on thermophilic strand displacement amplification (tSDA). Primers (sequences shown in SEQ ID No.37-40) and a probe (SEQ ID No. 41) are included.

The diagnostic kits can be:

1) 1) Double-antibody sandwich ELISA kits, including the solid substrate, capturers immobilized on the solid substrate, biotinylated capturers and enzymatic substrate (colorimetric). Capturers immobilized are monoclonal antibodies while biotinylated capturers are polyclonal antibodies. Or

2) Blotting kits including solid substrate, capturers, enzymatic labeled secondary antibody and enzymatic substrate for colorimetric detections. The capturers are monoclonal antibodies and biotinylated capturers are polyclonal antibodies. Or

3) Competitive ELISA kits including solid substrate, immobilized antigen, biotinylated capturers, the enzymatic substrate for colorimetric detections and specific monoclonal antibodies. The biotinylated capturers are polyclonal antibodies.

Positive and negative control and a blank sample are included in the diagnostic kit.

As components of the double-antibody ELISA kit, the monoclonal antibody is rat-anti-cystatin S antibody; the solid substrate is an ELISA plate and the biotinylated polyclonal antibody is biotinylated rabbit-anti-cystatin S polyclonal antibody.

Or he mentioned testing kits are based on double-antibody ELISA kits, wherein the solid substrate is an ELISA plate; the capturer immobilized on the solid substrate is rat-anti-cystatin S monoclonal antibody (R&D, MAB 1926, 5 μg/mL); the biotin labeled polyclonal antiboy is biotinylated rabbit-anti-cystatin S polyclonal antibody.

Or the testing kits are based on competitive ELISA kit, wherein the solid substrate of the assay is the ELISA plate; the concentration of cystatin S is 5 μg/mL; the monoclonal antibody is rat-anti-cystatin S antibody with valence of 1:2000; the enzymatic labeled secondary antibodies are ALP labeled goat-anti-mouse IgG with valence of 1:2000. The enzymatic substrate is ALP. The volume ratios of cystatin S and enzymatic labeled secondary antibody to the substrate are 1:2.

Or the testing kits based on immunoblotting, wherein the solid substrate is the nitrocellulose membrane; the capturers are rat-anti-cytatin S monoclonal antibodies with valence of 1:1000; the enzymatic labeled secondary antibodies are HRP-labeled goat-anti-rabbit IgG (Jackson ImmunoResarch); the enzymatic substrate for colorimetric detections are TMB solution (Kirkegaard and Perry Laboratories Inc. (Gaithersburg, Md.), “TMB Peroxidase Substrate” solution Cat.No. 50-76-01).

The mentioned protocol for the testing kits, wherein the details are described as following.

Coat the ELISA plate (Corning) with cystatin S (Abnova, Cat.No H00001472-P01, 5 μg/mL) and backfilled by 3% BSA. Samples with rat-anti-cystatin S monoclonal antibody (R&D, Cat.No MAB1296, valence 1:2000) and serum (8 times dilution) are prepared, which is incubated under 4° C. overnight. The sample is applied on the pre-treated ELISA plate and incubated for 1 hour under 37° C. Holes with samples are washed by TBS buffer (10 mM Tris-HCl, 154 mM NaCl, pH 7.5). ALP labeled goat-anti-mouse IgG (Jackson ImmunoRearch, valence 1:2000) dissolved in TBS with 0.3% BSA are applied in the holes and incubated for 1 hour under 37° C. Substrate of ALP (KPL, Blue Phos solution, Kirkegaard and Perry Laboratories Inc. (Gaithersburg, Md.), Cat.No. 508805) was applied and OD (405 nm) was read on a microplate reader.

The fourth objective of this invention is to provide an in vitro method for breast cancer diagnosis and testing kits for in vitro diagnosis of easy operation, high sensitivity and excellent specificity.

In order for the realization of the objective, the technologies of this invention is described in detail as following.

The expression level or the quantitative content of the breast cancer markers measured via the testing kits is compared with those of healthy people to decide whether the result is positive or not; or the result is read positive if it is higher than a cutoff value. The cutoff value is obtained through the comparison of the breast cancer markers expressions/levels in the body fluids or tissue samples of breast cancer patients and healthy people. The cutoff value is of statistical significance. Samples include one or more of the following: blood, urine, marrow, breast cancer cell lines, breast cancer tumors and tumor adjacent tissues and lymph node tissues. For instance, the cutoff value in this case in 3.434 ng/mL.

The testing kits for the prediction and diagnosis of breast cancer are kits for the measurements of cystatin S protein expression. The kit include solid substrate, capturers immobilized on the solid substrate, biotinylated capturers and enzymatic substrate for colorimetric detections. The capturers immobilized are monoclonal antibodies and the biotinylated capturers are polyclonal antibodies.

Or the testing kits for cystatin S protein expression measurement include solid substrate, cystatin S protein coated on the plate, rat-anti-cystatin S monoclonal antibodies, enzymatic labeled secondary antibodies and enzymatic substrate for colorimetric detections.

Or the testing kits for cystatin S protein expression measurement include solid substrate, capturers, enzymatic labeled secondary antibodies and enzymatic substrate for colorimetric detections. Capturers are monoclonal antibodies and biotinylated capturers are polyclonal antibodies.

Testing kits are based on double-antibody sandwich ELISA, wherein the solid substrate of the assay is the ELISA plate; capturers immobilized are rat-anti-cystatin S monoclonal antibody; the biotinylated capturers are rabbit-anti-cystatin S polyclonal antibody with valence of 1:1000. The enzymatic substrate is ALP.

Or the kits are based on competitive ELISA, wherein the solid substrate of the assay is the ELISA plate; the concentration of cystatin S is 5 μg/mL; the monoclonal antibody is rat-anti-cystatin S antibody with valence of 1:2000; the enzymatic labeled secondary antibodies are ALP labeled goat-anti-mouse IgG with valence of 1:2000. The enzymatic substrate is ALP. The volume ratios of cystatin S and enzymatic labeled secondary antibody to the substrate are 1:2.

Or the testing kits based on immunoblotting, wherein the solid substrate is the nitrocellulose membrane; the capturers are rat-anti-cytatin S monoclonal antibodies with valence of 1:1000; the enzymatic labeled secondary antibodies are HRP-labeled goat-anti-rabbit IgG; the enzymatic substrate for colorimetric detections are TMB solution.

Advantages of this invention are 1) the applications of the expression CST4 mRNA and cystatin S protein in the diagnosis of breast cancers, real time monitoring of the cancer development and breast cancer prognosis prediction are verified in this invention with large-scale sample. The results are of significant accuracy; the invention can be applied in the development of novel methods for breast cancer diagnosis and real time monitoring, as well as breast cancer prognosis predictions; 2) Testing reagents and kits with high sensitivity for breast cancer diagnosis and real time monitoring, as well as breast cancer prognosis predictions, are included in this invention.

### BRIEF DESCRIPTION OF THE FIGURES

FIG. 1. Recombinant plasmid Pmd18-T that involve CST4

FIG. 2. Expression of CST4 gene in normal tissues including tonsil, posterior pituitary, thyroid, salivary gland, skeletal muscle, bone marrow, peripheral blood without red blood cells and platelets, lung, stomach, liver, heart, kidney, adrenal gland, intestines, colon, pancreas, spleen, bladder, prostate, ovarian, uterus, placenta and testis), human breast cancer cell lines (HCC1937, SK-BR-3, and MCF-7) and normal breast tissue cell line (Hs578Bst).

FIG. 3. Expression comparison of CST124, CST1, CST2, CST4 in 20 cases of breast cancer tumors and their respective tumor adjacent tissues using real time qPCR with fluorescent dye as the probe.

FIG. 4. Expression of CST4 quantified by real time PCR in 100 cases of breast cancer tumors and their respective tumor adjacent tissues.

FIG. 5. Expression of CST4 quantified by real time PCR in 40 cases of breast cancer tumors and 40 biopsy samples.

FIG. 6. Expression of CST4 quantified by real time PCR in 30 lymph node sample with breast cancer metastasis and 30 lymph node sample without breast cancer metastasis.

FIG. 7. Comparison of accuracies for breast cancer diagnosis using cytological studies and CST4 expression in peripheral blood quantified by real time PCR.

FIG. 8. Comparison of accuracies for breast cancer marrow metastasis predictions using cytological studies and CST4 expression quantified by real time PCR.

FIG. 9. CST4 expression in cell-free RNA of 50 breast cancer patients, 30 healthy people and 30 mastitis patients quantified by real time PCR (A) and receiver operating characteristic (ROC) curve (B). ROC curve can be used for evaluation of the sensitivity and specificity of method to distinguish breast cancers from mastitis and normals.

FIG. 10. CST4 expression in cell-free RNA of 50 breast cancer patients, 30 healthy people and 30 mastitis patients quantified by LCR.

FIG. 11. CST4 expression in cell-free RNA of 50 breast cancer patients, 30 healthy people and 30 mastitis patients quantified by reverse transcription strand displacement amplification (rtSDA).

FIG. 12. CST4 expression in urine samples of 30 breast cancer patients, 20 healthy people and 20 mastitis patients quantified by nucleic acid based amplification (NASBA).

FIG. 13. CST4 expression by transcription-mediated amplification (TMA) of 80 breast cancer patients with various pTNM stages (30 I+II cases and 50 III+IV cases).

FIG. 14. Cystatin S expression in culter supernatant of breast cancer cell lines and serums of healthy people.

FIG. 15. Cystatin S expression in breast cancer cell lines and serums of healthy people.

FIG. 16. Cystatin S expression by competitive ELISA in serum samples of 30 breast cancer patients and 20 healthy people.

FIG. 17. Specificity and sensitivity comparison of cystatin S and CEA (measured by ELISA) for breast cancer prediction.

FIG. 18. Prognosis-free survival (PFS) of post-treatment breast cancer patients with higher or lower cystatin S expression than the median value.

### DETAILED DESCRIPTION

### 1. Molecular Detections

The molecular biological technologies mentioned above are illustrated by the following examples. It should be noted that these example are for clarification for this invention instead of limiting the applications of this invention. Protocols in Molecular Cloning: A Laboratory Manual (Edited by J. Sambrook et al) were strictly followed for experimental procedures if not noted. Or guidelines from the manufacturers were followed. If not mentioned, percentage and fraction are based on weight.

### Materials and Methods

All clinical samples are acquired from Beijing Friendship Hospital, with the regulations of the hospital strictly observed and consent forms signed by the patients.

Biopsy samples suspected as tumor or their adjacent tissues are compared. RNA in these samples as well as the lymph node samples should be immediately extrated after sample acquisition or they should be stored in liqui nitrogen or RNA later (Ambion).

Peripheral blood, marrow or urine samples are centrifuged for 20 minutes (4000 rpm, 4° C.). The supernatant is centrifuged for another 10 minutes (13000 rpm, 4° C.). Separate the supernatant and precipitation. RNA extraction should follow immediately or the samples are stored under −20° C. or −80° C.

### Real Time PCR Using TaqMan Probes:

samples are submitted for nucleic acids extraction using commercially available kits. A non-limiting example is phenol-chloroform extraction. All RNA in the samples is obtained following the Trizol kit manufactured by Invitrogen. The quality of the RNA extracted is examined following protocols in references such as Molecular Biology Experiments (by J. Li). Reverse transcription of mRNA is realized by commercially available kits and the manuals are followed. cDNA solutions are prepared with appropriate concentration gradients. Primers for biochemical reactions are properly optimized. The primers for CST4 are copied from exon 1. Recombinant plasmid with CST4 amplicon are commercially available from Pegm-T (Promega, as shown in FIG. 1). Primers are copied from exons 1 and 3. The PCR process is monitored by the hydrolysis of the TaqMan probe.

The optimized sequences for primers of CST4 are: gctctcaccctcctctcctg (SEQ ID No.1) and tatcctattctcctccttgg (SEQ ID No.2). The sequence of the probe is 5′-fam-ctccagctttgtgctctgcctctg-tamra-3′ (SEQ ID No.3). The size of the amplicon is 142 bases pairs.

The optimized sequences for primers of recombinant plasmid that contains CST4 amplicon are tgcctcgggctctcaccctcctct (SEQ ID No.22) and tgggtggtggtcggtgtgactggc (SEQ ID No.23).

In a typical testing, the sample, positive and negative controls and recombinant plasmid standards are amplified simultaneously. Cross points (CP) of the recombinant plasmid standards with appropriate concentration gradients versus concentration are plotted and a calibration curve is obtained. Copies of the gene expression in the sample and controls are quantified through the curve.

### Real Time PCR Using Fluorescent Dyes as the Probe:

pre-treatments fo the samples are identical to what is described in the section of real time PCR using TaqMan probes. The sequences for primers for the amplifications of CST1, CST2 and CST4, which are amplified simultaneously, are agtcccagcccaacttgga (SEQ ID No.24) and gggaacttcgtagatctggaaaga (SEQ ID No.25). The sequences of the primers for CST4 amplifications are agtacaacaaggccaccgaagat (SEQ ID No.4) and agaagcaagaaggaaggagggag (SEQ ID No. 5), or tacaacaaggccaccgaagatga (SEQ ID No.6) and agaagcaagaaggaaggagg gag (SEQ ID No.7), or tgctactcctgatggctaccctg (SEQ ID No.8) and gtggccttgttgtactcgctgat (SEQ ID No. 9), or agtacaacaaggccaccgaagat (SEQ ID No.10) and taccaggtctattagaagcaagaagga (SEQ ID No. 11), or tgctactcctgatggctaccctg (SEQ ID No.12) and catcttcggtggccttgttgtac (SEQ ID No. 13), or tgctactcctgatggctaccctg (SEQ ID No.14) and tactcatctt cggtggccttgtt (SEQ ID No. 15), or tgggattatcctattctcctccttg (SEQ ID No.16) and ctccagcttt gtgctctgcctct (SEQ ID No. 17), or tgctactcctgatggctaccctg (SEQ ID No.18) and ctcatcttcg gtggccttgt tgt (SEQ ID No. 19), or tacagtgggtgggagtgggtggt (SEQ ID No.20) and gagtgggtac agcgtgccct tca (SEQ ID No. 21). The sequences of the primers for CST2 amplifications are cagaagaaacagttgtgctc (SEQ ID No.26) and ggagtaggaggtggtcag (SEQ ID No.27). The sequences of the primers for CST1 amplifications are tctcaccctcctctcctg (SEQ ID No.28) and ttatcctatcctcctccttgg (SEQ ID No.29). β-actin is applied as an internal reference. The sequences of the primers for β-actin amplifications are aagatcattgctcctcctg (SEQ ID No.30) and cgtcatactcctgcttgc (SEQ ID No.31). Genes in cancerous tumors, tumor adjacent tissues (TAT) and internal reference gene are amplified simultaneously. The copies of the genes are quantified by the following equation, wherein ct is the cycle number when the fluorescence signal is over the background. SYBR Greenk, Eve Green, LC Green, etc are among fluorescent dyes that apply

$${Expression}_{relative} = \frac{{2}^{{ct}_{tumor} - {ct}_{ref}}}{{2}^{{ct}_{TAT} - {ct}_{ref}}}$$

### In Vitro RNA Amplification by Nucleic Acid Based Amplification (NASBA):

The kits include T7 RNA polymerase, RNase H, avian myeloid leukemia virus (AMV) reverse transcriptase, ribonucleotide triphosphate (NTP), deoxyribonucleotide triphosphate (dNTP), primers for CST4 amplifications which identical as mentioned in the section of “real time PCR using TaqMan probes”, fluorescent dyes for RNA amplification monitoring (Ribo-Green fluorescent dye). The RNA template is amplified by 2⁹-2¹² fold after 2 hours incubation under 42° C. The fluorescence of the amplified product was monitored for the quantification of the template concentration before the amplification.

### Example 1

### Expression of CTS4 and Other Members of the CST Superfamily

### 1. CST4 in Various Human Tissues

All tissue samples were purchased except the breast tissue which was acquired from Beijing Friendship Hospital (BFH). CST4 mRNA expression in various human tissue samples was measured on HG-U95AV Human GeneChip Array (Affymetirx); protocols on the user manual are followed. Quantifications of CST1 mRNA expressions were realized by the β-actin fluorescence calibration curve.

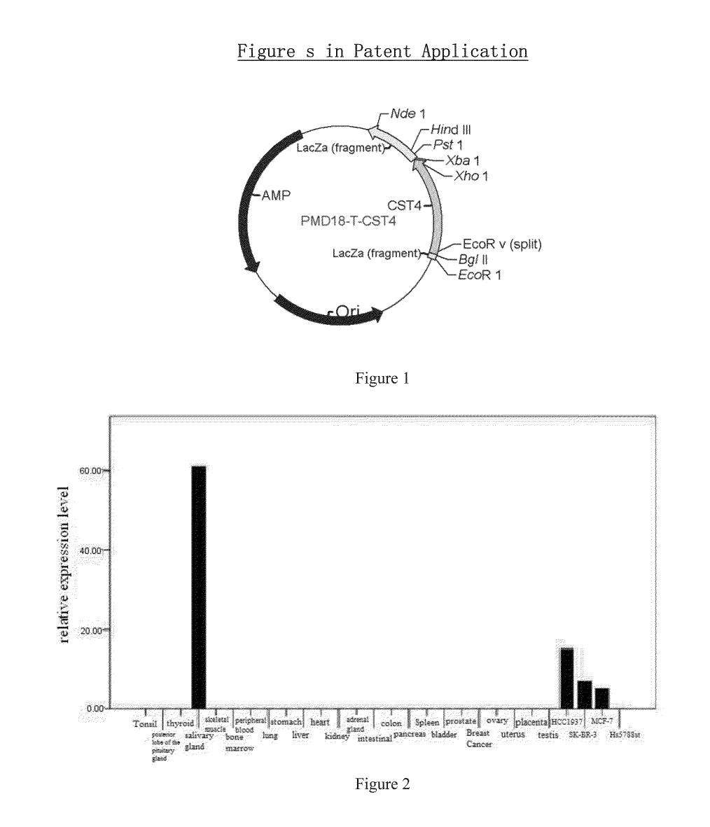

As shown in FIG. 2, CST4 expression is only high in salivary gland. No CST4 expression was observed in other tissues tested. The result indicates that CST4 is a good candidate for pathological diagnosis because of its low background signal. CST4 is over-expressed in HCC1937, SK-BR-3 and MCF-7, which are breast cancer cell lines and not expressed in Hs578Bst, which is normal breast tissue cell lines. It is concluded that CST4 can be used as a marker for breast cancer diagnosis.

2. Expression of mRNA of CST4, CST124, CST1 and CST2 in Breast Cancerous Tumors and Tumor Adjacent Tissues.

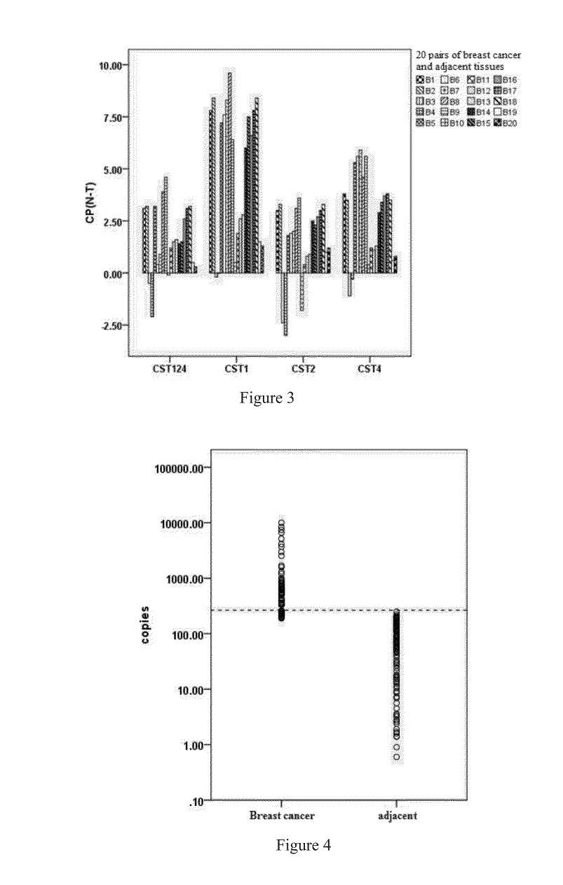

Expression of mRNA of CST4, CST124, CST1 and CST2 in twenty pairs (numbered as C1, C2 . . . C20) of breast cancer tumors and their respective adjacent tissues were compared. It was discovered that CST4 expression difference in tumors and their adjacent tissues is significant, larger than all genes except CST1 (FIG. 3). All samples were diagnosed pathologically with breast cancer. Real time PCR using fluorescent dye as the probe was used for the quantification of the genes expression, which was verified by simultaneous amplifications of positive and negative control samples. The results of the controls met the expectation.

The testing kit based on real time PCR with fluorescent dyes as the probe for mRNA expression quantification contains:

1) Primers for CST4 amplification, whose sequences are shown as following:

(SEQ ID No. 4)
Upstream primer: agtacaacaa ggccaccgaa gat
(SEQ ID No. 5)
Downstream primer: agaagcaaga aggaaggagg gag
or
(SEQ ID No. 6)
Upstream primer: tacaacaagg ccaccgaaga tga
(SEQ ID No. 7)
Downstream primer: agaagcaaga aggaaggagg gag
or
(SEQ ID No. 8)
Upstream primer: tgctactcct gatggctacc ctg
(SEQ ID No. 9)
Downstream primer: gtggccttgt tgtactcgct gat
or
(SEQ ID No. 10)
Upstream primer: agtacaacaa ggccaccgaa gat
(SEQ ID No. 11)
Downstream primer: taccaggtct attagaagca agaagga
or
(SEQ ID No. 12)
Upstream primer: tgctactcct gatggctacc ctg
(SEQ ID No. 13)
Downstream primer: catcttcggt ggccttgttg tac
or
(SEQ ID No. 14)
Upstream primer: tgctactcct gatggctacc ctg
(SEQ ID No. 15)
Downstream primer: tactcatctt cggtggcctt gtt
or
(SEQ ID No. 16)
Upstream primer: tgggattatc ctattctcct ccttg
(SEQ ID No. 17)
Downstream primer: ctccagcttt gtgctctgcc tct
or
(SEQ ID No. 18)
Upstream primer: tgctactcct gatggctacc ctg
(SEQ ID No. 19)
Downstream primer: ctcatcttcg gtggccttgt tgt
or
(SEQ ID No. 20)
Upstream primer: tacagtgggt gggagtgggt ggt
(SEQ ID No. 21)
Downstream primer: gagtgggtac agcgtgccct tca
Primers for the amplification of β-actin as an
internal reference
(SEQ ID No: 30)
Upstream primer: aagatcattgctcctcctg
(SEQ ID No: 31)
Downstream primer: cgtcatactcctgcttgc

2) Reagents for nucleic acids extractions and reverse transcriptions, SYBR Green fluorescent dye, dNTP, Taq polymerase, RNAse-free water, standard solutions, positive and negative control samples, 10× buffer and magnesium chloride solution.

### Example 2

### CST4 Expression in Various Tissues and Locations

### 1) CST4 Expression in Breast Cancer Tumors and Tumor Adjacent Tissues.

Testing kits for CST4 mRNA expression based on real time PCR with TaqMan probes. The kit contains the following.

1)Primers and probe:
(SEQ ID No. 1)
Upstream Primer: gctctcaccctcctctcctg
(SEQ ID No. 2)
Downstream Primer: tatcctattctcctccttgg
(SEQ ID No. 3)
Probe: 5'-fam-ctccagctttgtgctctgcctctg-tamra-3′

2) Reagents for nucleic acids extractions and reverse transcriptions, SYBR Green fluorescent dye, dNTP, Taq polymerase, ribonuclease-free water, standard solutions, positive and negative control samples, recombinant plasmid samples with CST4 gene, 10× buffer and magnesium chloride solution.

All samples were diagnosed with breast cancer before RNA extraction and reverse transcription, by which cDNA was obtained. Real time PCR was applied for the quantification of the expression of CST4 in breast cancer tumors and their respective adjacent tissues. One hundred samples were tested in this trial. The linearity of the calibration curve and amplification yield met the respective requirements. Results for the amplification of the positive and negative control samples were of expectation. No amplification for samples without the template was observed.

As shown in FIG. 4, CST4 mRNA expression is high in pathologically diagnosed malignant breast cancer tumors and low in their respective adjacent tissues. The median value of the CST4 expression in tumors is 7.08 fold higher than in adjacent tissues, which indicates that CST4 mRNA is an excellent marker for breast cancers. Cancerous tissues can be distinguished from the normal tissues if 264.92 copies is the cutoff value, which is a proposed reference for the clinical diagnosis of breast cancers.

2) CST4 Expression in Biopsy Samples from Breast Cancer and Mastitis Patients

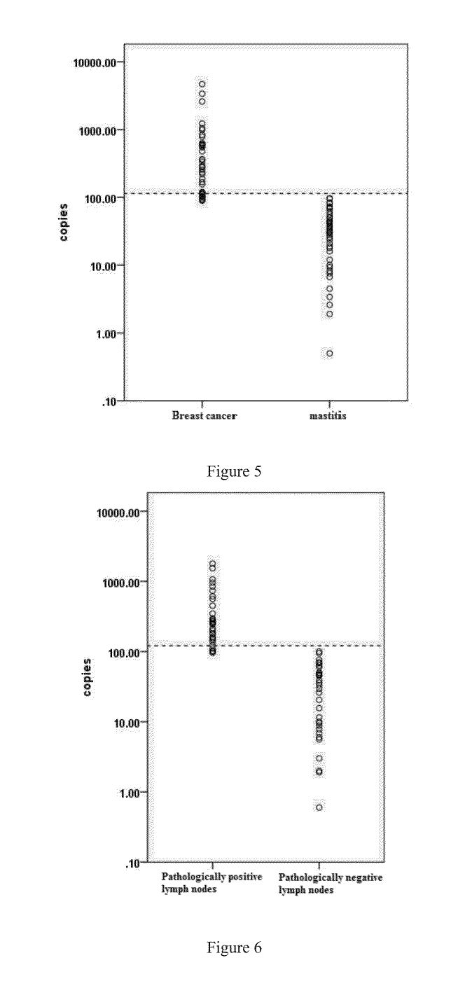

Biopsy samples are of great difference from surgical sample in that the percentage of tumor cells varies. The tumor tissue is sometimes a very small fraction of the whole biopsy sample, or no tumor tissues are included in the biopsy samples. The inventors tested and compared the CST4 expression in 40 breast biopsy samples from breast cancer patients and 40 biopsy samples from mastitis patients. It was discovered that the median of CST4 expression in cancerous samples was 9.15 fold higher than in mastitis samples. Cancerous tumor can be distinguished from inflammations if 113.795 is the cutoff value, which provides reference for breast cancer diagnosis using biopsy samples. The results are summarized in FIG. 5. Real time PCR was applied for gene expression quantification. The linearity of the calibration curve and amplification yield met the respective requirements. Results for the amplification of the positive and negative control samples were of expectation. No amplification for samples without the template was observed.

3. CST4 Expression in Lymph Node Sample with and without Breast Cancer Metastasis

Thirty surgical samples of lymph nodes with pathologically diagnosed breast metastases of varied size and thirty lymph node samples from patients with early-stage non-metastatic breast cancer were obtained. Early-stage cancer patients were selected to avoid undetectable lymph node metastasis and the resulting artifacts. Real time PCR was applied for the quantification of the expressions of CST4 and the detailed experimental procedures were identical as what was described in example 2(CST4 expression in breast cancer tumors and tumor adjacent tissues). The linearity of the calibration curve and amplification yield met the respective requirements. Results for the amplification of the positive and negative control samples were of expectation. No amplification for samples without the template was observed.

As shown in FIG. 6, CST4 expression is high in those samples with breast cancer metastasis while it is relatively low in those without cancerous metastasis. Median of CST4 expression in samples with metastasis is 8.458 fold higher than in samples without breast cancer metastasis. Cutoff value of 120.66 copies is possible for the distinguishing of metastasis. Two positive cases of CST4 expression were reported in the group of non-metastasis. These samples were carefully studied and micro-metastasis was discovered in both. If these two cases are considered as metastatic samples, CST4 mRNA expression testing is able to distinguish all metastatic cases in the trial. Micro-metastasis which is beyond the capacity of traditional pathological studies was discovered, indicating its higher sensitivity.

### 4. Accuracy Comparison of CST4 Expression Measurement by Real Time PCR and Cytological Studies for the Detections of Circulating Breast Cancer Cells in Peripheral Blood.

RNA was extracted from peripheral blood without red blood cells and platelet; CST4 mRNA expression was quantified by real time PCR, which was compared with samples from mastitis patients and healthy people to decide the existence of circulating breast cancer cells. The results were compared with cytological studies.

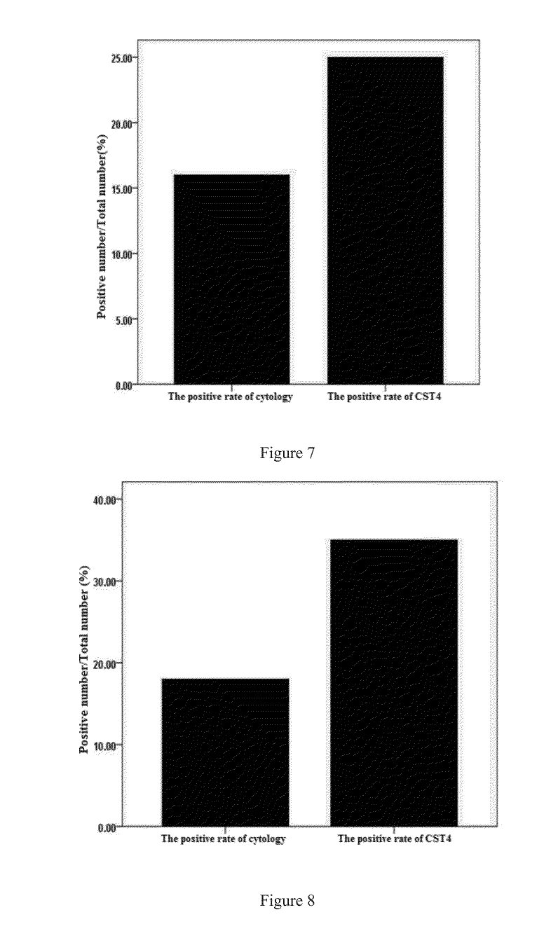

As summarized in FIG. 7, CST4 expression method is able to detect all cancerous cases as diagnosed by cytological studies. Cancer metastasis was discovered in cytological negative patients, evidencing that methods mentioned in the invention is more sensitive than cytological methods and micro-metastasis which is beyond what cytological methods can do can also be detected.

### 5. Marrow Metastasis Detected by Real Time PCR and Cytological Studies

CST4 mRNA of biopsy marrow samples from breast cancer patients was quantified by real time PCR. The results were compared with normal marrow samples for the detections of metastasis or micro-metastasis. The conclusions from these tests were compared with cytological studies.

As presented in FIG. 8, 95% samples with marrow metastasis (based on cytological studies) can be detected by CST4 mRNA testing. The higher positive rate than the cytological studies indicates better sensitivity.

### Example 3

### CST4 Expression in Breast Cancer Patients, Mastitis Patients and Healthy People

1. CST4 Expression in Serum Cell-Free RNA of Breast Cancer Patients, Mastitis Patients and Healthy People

Plasma samples from breast cancer patients (50 cases), mastitis patients (30 cases) and healthy people (30 cases) were collected. Cell-free RNA was extracted through commercial kits; real time PCR was used for the quantifications of CST4 expression.

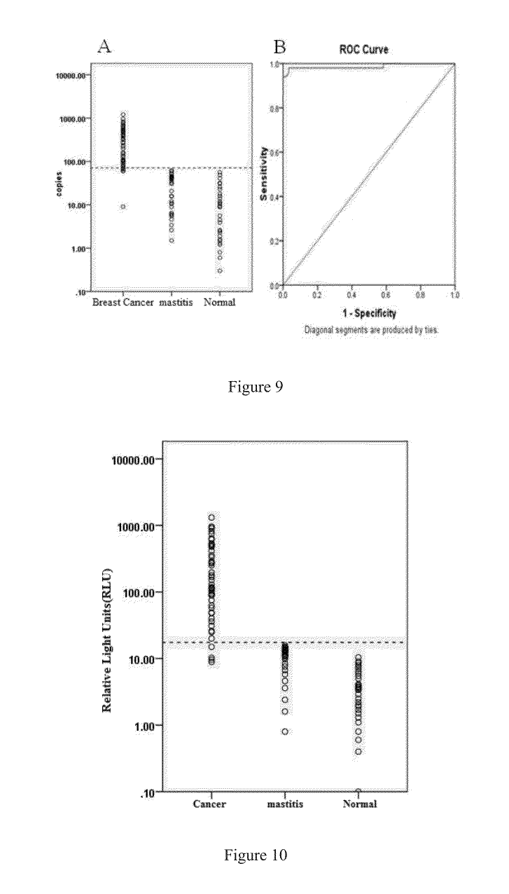

It was discovered that median of CST4 expression of the cancerous group is 8.87 fold and 25.62 higher than the inflammation group and normal group respectively (FIG. 9A). A cutoff value of 71.218 is able to distinguish the cancerous sample from non-cancerous samples. Receiver operating characteristic (ROC) curve of CST4 expression test as a method for breast cancer diagnosis is presented in FIG. 9B. High sensitivity and specificity are concluded by the integration of the curve of 0.987. CST4 is a specific marker for non-invasive breast cancer diagnosis by plasma samples.
2. CST4 Expression in Serum Cell-Free RNA of Breast Cancer Patients, Mastitis Patients and Healthy People Tested by Ligase Chain Reaction (LCR)

The testing kit for CST4 mRNA expression contains the following.
1) Four probes with hapten labeling: gggctctggcctcgagctccaagga (SEQ ID No.33), ataggataatcccaggtggcatctatgatg (SEQ ID No.34), tctcctccttggagctcgaggccagagccc (SEQ ID No.35) catcatagatgccacctgggattatcctat (SEQ ID No.36).
2) Commercially available reagents for nucleic acid extractions and reverse transcriptions. Other reagents are identical to LCx kit (Abbott Laboratories)

Cell-free RNA was extracted from plasma samples of breast cancer patients (50 cases), mastitis patients (30 cases) and healthy people (30 cases). The expression of mRNA of CST4 was tested by LCR method. As shown in FIG. 10, the median of the relative light units (RLU) of the cancerous samples is 10.881 and 35.286 fold higher than samples with inflammation and normal samples respectively. Breast cancer samples can be distinguished with a cutoff value of 17.458 RLU.
3. CST4 Expression in Serum Cell-Free RNA of Breast Cancer Patients, Mastitis Patients and Healthy Subjects Tested by Reverse Transcription Strand Displacement Amplification (rtSDA)

Testing kit for CST4 mRNA expression quantification based on tSDA contains the following.

1) CST4 B1 Primer:
(SEQ ID No. 37)
cccggcctctgtgtaccctgcta
CST4 S1 Primer:
(SEQ ID No. 38)
gaa-ctcgagctaccctggctggggctctgg
CST4 B2 Primer:
(SEQ ID No. 39)
ggtggccttgttgtactcgctgat
CST4 S2 Primer:
(SEQ ID No. 40)
gct-ctcgag agtgaagggcacgctgtac [symbol]
:
(SEQ ID No. 41)
5′-³²P-ttactcgag ctccaaggaggagaatagga-3′

2) Reagents for nucleic acid extractions and reverse transcriptions, dCTPαS, dATP, dGTP, dTTP, Bsob I and exo-Bca.

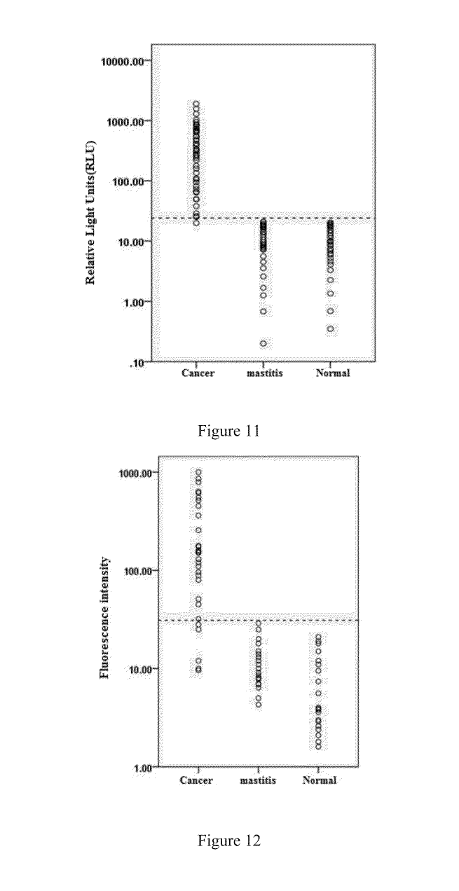

Cell-free RNA was extracted from plasma samples of breast cancer patients (50 cases), mastitis patients (30 cases) and healthy people (30 cases). The expression of mRNA of CST4 was tested by thermophilic strand displacement amplification (tSDA). As shown in FIG. 11, the median of the relative light units (RLU) of the cancerous samples is 34.58 and 35.89 fold higher than samples with inflammation and normal samples respectively. Breast cancer samples can be distinguished with a cutoff value of 24.095 RLU.
4. CST4 Expression in Urine Cell-Free RNA of Breast Cancer Patients, Mastitis Patients and Healthy People

Testing kit for CST4 mRNA expression quantification based on nucleic acid based amplification (NASBA) contains the following.

1)Primers and probes for CST4:
Upstream primer:
(SEQ ID No. 32)
aattctaatacgactcactataggg-gctctcaccctcctctcctg
Downstream primer:
(SEQ ID No. 2)
tatcctattctcctccttgg
Molecular beacon probe:
(SEQ ID NO. 3)
5′-fam-gcggcctccagctttgtgctctgcctctggccgc-dabsyl-
3′

2) Reagents for RNA extraction and revers transcription, T7 RNA polymerase, RNase H, avian myeloid leukemia virus (AMV) reverse transcriptase, ribonucleotide triphosphate (NTP), deoxyribonucleotide triphosphate (dNTP) and RNA fluorescent dye (Ribo-Green fluorescent dye).

Cell-free RNA was extracted from urine samples of breast cancer patients (30 cases), mastitis patients (20 cases) and healthy people (20 cases). The expression of mRNA of CST4 was tested by NASBA. As shown in FIG. 12, the median of the relative light units (RLU) of the cancerous samples is 15.86 and 38.35 fold higher than samples with inflammation and normal samples respectively. Breast cancer samples can be distinguished with a cutoff value of 30.92 RLU. CST4 is therefore an excellent marker in non-invasive urine test for breast cancer diagnosis

### Example 4

### CST4 as a Marker for Breast Cancer pTNM Staging, Real Time Monitoring of Tumor Development During Treatment and Breast Cancer Prognosis Preditions

Testing kit for CST4 mRNA expression quantification based on transcription-mediated amplification (TMA) contains the following.

1)Primers and probe for amplification:
Upstream primer:
(SEQ ID No. 32)
aattctaatacgactcactataggg-gctctcaccctcctctcctg
Downstream primer:
(SEQ ID No. 2)
tatcctattctcctccttgg
Molecular beacon probe:
(SEQ ID NO. 3)
5′-fam-gcggcctccagctttgtgctctgcctctggccgc-dabsyl-
3′

2) Any reagents included in Gen-probe TMA assay except the primers and probe.
1. CST4 Expression and Breast Cancer pTNM Staging

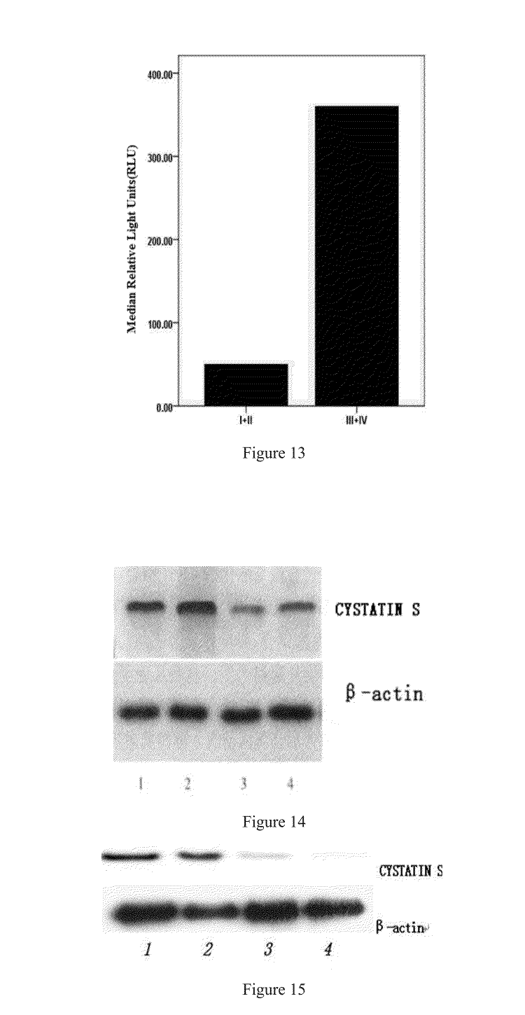

Cell-free RNA in plasma samples from 80 breast cancer patients (30 cases of I+II stages and 50 cases of III+IV stages) was extracted by commercial kits. CST4 expression was measured using TMA (transcription-mediated amplification) method. As presented in FIG. 13, RLU median of the late stage group (stages III+IV) is 7.2 fold higher than the early stage group (stages I+II). The results points that CST4 is a good indicator for breast cancer stages and can be sued for cancer stage determination.
2. Applications of CST4 Expression in the Real Time Monitoring in Breast Cancer Treatment

Serum CST4 expression of breast cancer patients taking therapies (6 patients with chemotherapy and 4 patients with radiotherapy) was monitored by real time PCR. The tumor development was compared and correlated with CST4 expression level in blood.

As summarized in Table 1, CST4 expression decreases for patients with effective therapy, which was evidenced by the decreased size of the tumors. CST4 expression increases with the continuation of the therapies for patients with ineffective therapy, which was evidenced by the increased size of the tumors. CST4 is thus proposed as a marker for real time monitoring of therapy effects.

TABLE 1
Real time CST4 expression of in breast cancer patients blood during treatment
by real time PCR

| | | CST4 | | | | | |
|---|---|---|---|---|---|---|---|
| | | expression (copy) | | | Tumor Size (cm) | | |
| | | Cycle1 | Cycle2 | Cycle3 | Cycle1 | Cycle 2 | Cycle3 |
| Effective | Chemo1 | 781.32 | 521.78 | 80.64 | 2.5 | 1.5 | <1 |
| | Chemo3 | 1533.6 | 1314.6 | 125.25 | 3.5 | 2.5 | <1 |
| | Chemo5 | 1213.5 | 439.89 | 66.64 | 3 | 2 | <1 |
| | Radio2 | 1434.58 | 1160.33 | 116.83 | 3 | 1.5 | 1 |
| | Radio3 | 2062.66 | 1689.44 | 189.65 | 3 | 2.5 | 1.5 |
| Ineffective | Chemo2 | 1466.14 | 1984.62 | 2433.57 | 3 | 3 | 3.5 |
| | Chemo4 | 956.92 | 1156.34 | 1846.21 | 2 | 2 | 2.5 |
| | Chemo6 | 646.2 | 826.7 | 1032.55 | 1 | 1 | 1.5 |
| | Radio1 | 1032.4 | 1246.8 | 1989.61 | 2 | 2.5 | 3 |
| | Radio4 | 936.4 | 1048 | 1678 | 1 | 1.5 | 2 |

3. CST4 Expression for Breast Cancer Prognosis Predictions

The blood CST4 expression of five post-treatment breast cancer patients was monitored after 1 month, 3 months and 1 year after the treatment by quantitative real time PCR. As shown in Table 2, two patients have cancerous recurrence. Increasing CST4 expression was observed with these two patients. The cancer recurrences were not detected until the CSTT4 expression reached around 1000 copies. The other three patients did not have cancer recurrence and on significant CST4 expression increase was observed with them. Thus CST4 is a good marker for cancer prognosis predictions.

TABLE 2
CST4 Expression Monitoring of Post-treatment Breast Cancer Patients by
quantitative real time PCR

| | | CST4 (copy) | | | Tumor Size (cm) | | |
|---|---|---|---|---|---|---|---|
| | | 1 month | 3 monthhs | 1 year | 1 months | 3 monthhs | 1 year |
| Cancer | Patient 1 | 56.84 | 198.87 | 1135.24 | ND | ND | 1 |
| Recurrence | Patient 5 | 15.25 | 64.34 | 786.59 | ND | ND | <1 |
| No Cancer | Patient 2 | 23.6 | 40.5 | 36.3 | ND | ND | ND |
| Recurrence | Patient 3 | 52.43 | 39.8 | 48.65 | ND | ND | ND |
| | Patient 4 | 67.9 | 79.6 | 86.79 | ND | ND | ND |

### Part II Protein As Marker

Non-invasive protein probes, testing kits, methods and protocols for breast diseases diagnosis, monitoring and therapy evaluation are discussed in the following passages of this invention.

Recombinant cystatin S protein was purchased from Abnova (0.06 μg/μL, Cat. No. H00001472-P01). Rat-anti-cystatin S monoclonal antibody was purchased from R&D with valence of 1:2000 (Cat. No. MAB1296). Rabbit-anti-cystatin S polyclonal antibody was purchased from Abcam with valence of 1:1800 (Cat. No. ab58515).

This invention provides a method for the determination of breast tissue condition and prediction of breast cancer recurrence and metastasis. The evaluation of the therapies can be realized by methods described in this invention. This method measures at least one protein concentrations in samples provided by the patient. Cystatin S protein and its quantitative or semi-quantitative determination in the sample are of great favor. Although various molecules were reported for protein detection, specific antibodies or their fragments of cystatin S, are preferred in this invention. The methods detection protocols and testing kits can be used for breast cancer screening of people without cancerous symptoms.

At least one or more (preferred) antibodies or fragments that bind specifically to at least one epitope of cystatin S are applied in the detection mentioned in this invention. The antibodies can be monoclonal or polyclonal. The preferred monoclonal antibody binds cystatin S with the sequence shown in SEQ ID No.50. The antibody is acquired with cystatin S as immunogen. The immunogen concentration is preferred contant. Therefore, this invention provides a method for cystatin S quantification by the immune-response of the antibodies to the presence of cystatin S.

Subjects in this invention are human beings, wherein the immune-response in the test features antibodies and human peptides. The immune-reactions might be measured by any proper methods, which include but not limited to: ELISA, immune-blotting or the combination of both. Competitive ELISA and double-antibody sandwich ELISA.

The monitoring and diagnosis of breast tissue conditions, the monitoring of the effect of the therapies for breast diseases can be quatified by the immune-reaction via the quantification of cystatin S expression.

We claim in this invention all testing kits for the detection of the marker (cystatin S and its epitope herein) in the samples. The condition of the breast tissue and breast diseases can be diagnosed through the kits. The monitoring of breast disease therapies and prediction for cancer recurrence and metastasis are among the applications of the kits, too. Anti-cystatin S antibodies or their fragments should be included in the kits. These antibodies or their fragments bind cystatin S in fluidic samples such as serum. The binding event of the antibody or its fragement and cystatin S should be monitored or detected by a reporting unit.

The reporting unit of the preferred or optimized kit can be antibodies or their fragments with functional labeling. Herein, the reporting unit is preferred as appropriate IgG or IgM antibody. The labels can be and preferred as enzymes that catalyze reactions with substrate color change such as peroxidase. The labels are preferred to be covalently conjugated to the secondary antibody. Or, the labels can be fluorescent dye.

Testing kit based on ELISA is preferred in this invention.

The ELISA testing kits mentioned are competitive ELISA kit or double-antibody sandwich ELISA kit, whose details are described next. The antibodies or its fragment are incubated with the analyte. The antibody is a monoclonal antibody. Cystatin S protein, which is the immunogen for the production of anti-cystatin S antibody, is conjugated with a microplate (solid substrate herein). The pre-incubated mixture is applied on the ELISA plate; unbound antibodies then bind to the antibodies immobilized on the plate. The reporting unit is the immunoglobulin especially IgG and IgM, which is capable of detecting the antibodies on the plate. It should be noted that the antibodies are conjugated with the enzymes or fluorescent labeling for detections.

Another methodology of this invention is immune-blotting, or Western Blot, wherein the proteins in the sample are separated by gel electrophoresis such as PAGE and then transferred to solid substrate such as nitrocellulose membrane. One of the transfer methods is the electro-transfer. The analyte interacts with its specific antibody (monoclonal antibody preferred). The immune-reaction can be monitored by appropriate methods such as enzymatic/fluorophore labeled anti-antibodies.

Another preferred methodology in this invention is testing kits based on affinity column. In a typical process, the antibody or its fragment is immobilized on the column, and the sample solution passes the column slowly. The antibodies herein are monoclonal or polyclonal. Antibodies mentioned above are preferred.

The sample solution passes the column, when the analyte protein interacts with the immobilized antibodies and remains in the column. The analyte protein is then eluted by applying competitive antigen for the antibodies or by changing the running buffer conditions. If multiple proteins are to be analyzed, it is preferred to elute the proteins at different time. The proteins can be quantified by various methods which are well known, such as UV absorbance detection.

Cystatin S testing kits are provided in this invention. Two-channel cystatin S indicator accurately tells whether the cystatin S level is higher than normal or not. Fluidic samples are preferred for the testing. The kit includes containers for the sample, antibodies and their fragments and indicators. Monoclonal antibodies are of favor, especially the antibodies mentioned above. All solutions and buffers necessary for the testing and manuals for operations and data interpretation should be included in the kit. The kit should be carried on by professionals in any locations such as hospitals, clinics and houses.

Testees for this invention can be people with breast uncomforts. The test includes early screening of breast cancers for the testee.

Testees for this invention can be people with mastitis. The test includes early screening of breast cancers for the testee.

Testees for this invention can be people with family history of breast cancer. The test include early screening of breast cancers for the testee.

Samples to apply this invention include but not limited to serum, plasma, urine and blood, which includes whole blood or its fractions

As mentioned above, antibodies in this invention can be used for the diagnosis of breast disease and prediction for the recurrence and metastasis of breast cancer. Well known methods in this field can be combined with the kit. For instance, fluorescence method can be combined with the kit to test the cystatin S level in plasma, serum or urine, and then the existence of a specific disease.

The purpose, advantages and features of this invention are revealed in the following examples. Besides, experimental details that support our claims and conclusions made above are also included in the following examples.

### Materials and Methods

Recombinant cystatin S protein was purchased from Abnova (0.06 μg/μL, Cat. No. H00001472-P01).

Antibodies: Rat-anti-cystatin S monoclonal antibody was purchased from R&D with valence of 1:2000 (Cat. No. MAB1296). Rabbit-anti-cystatin S polyclonal antibody was purchased from Abcam with valence of 1:1800 (Cat. No. ab58515).

Immuno-precipitation: 2 mM Phenylmethanesulfonyl fluoride (PMSF), staphylococcal protein A immobilized agarose gel and cystatin S antibody are added in the sample. The mixture is gently stirred under 4° C. overnight. Dimethl pimelimidate is applied to conjugate the anti-cytatin S antibody to the agarose gel. The precipitates are washed and treated with N-glycosidase F and the protein is purified by SDS-PAGE.

Before the application of N-glycosidase F, the precipitate is boiled in 10 μL citrate buffer (50 mM, pH 6.0, 0.5% SDS). 10 μL phosphate buffer (200 mM, pH 8.0 with 40 mM EDTA) and N-octyl glucoside (3%) and N-xylanase are added in the mixture (40 mU), which is incubate overnight (37° C.). Loading buffer are then added for SDS-PAGE purification. Unless noted elsewhere, 15% polyacrylamide gel is used for PAGE. The gel is imaged by 20% 2,5-diphenyl oxazole solution.

Protein electro-transfer and immune-blotting: Transfer the protein on the nitrocellulose membrane, which is incubated in PBS buffer with 5% skimmed milk power and 0.1% Brij-35 for 2 hours (ambient temperature). The membrane is then incubated in rabbit-anti-cystatin S polyclonal antibody solution overnight (4° C.). The membrane is washed by PBS buffer (with 0.1% Brij-35) for three times. It is incubated with peroxidase labeled goat-anti-rabbit IgG solution (0.27 μL) for an hour (37° C.), followed by four times washing by PBS buffer (with 0.1% Brij-35) and one time washing of PBS. The membrane is imaged by commercial TMB solution (TMB Peroxidase Substrate, Kirkegaard and Perry Laboratories Inc. (Gaithersburg, Md.) Cat.No. 50-76-01). Or its tested by ELC method, during which the membrane is soaked in a solution with 5.4 mM hydrogen peroxide solution, 2.5 nM luminol and 400 mM p-coumaric acid (dissolved in 100 mM Tris-HCl, pH 8.5) and imaged on Agfa CP-BU foil.

Competitive ELISA: ELISA plate (Corning) is coated by cystatin S solution (5 μg/mL) and backfilled by 3% BSA solution. Eight serum samples (2× diluted) and polyclonal rat-anti-cystatin S antibody (with valence of 1:1000) are incubated overnight (4° C.) and applied on the pre-treated ELISA plate. The plate is incubated for one hour under 37° C. Samples holes are washed by TBS buffer (10 mM Tris-HCl, 154 mM NaCl, pH 7.5). Add alkaline phosphatase (ALP) labeled goat-anti-mouse IgG (Jacksonwi ImmunoResearch with valence of 1:2000) solution was added and incubated for an hour (37° C.). TMB solution (TMB Peroxidase Substrate, Kirkegaard and Perry Laboratories Inc. (Gaithersburg, Md.) Cat.No. 50-76-01) is added and OD at 405 nm is quantified by a microplate reader.

Double-antibody Sandwich ELISA: ELISA plate (Corning) is coated by monoclonal rat-anti-cystatin S solution (5 μg/mL) and backfilled by 3% BSA solution. Eight serum samples (2× diluted) are incubated in the holes of the plate for an hour (37° C.). The plate is washed by TBS buffer (10 mM Tris-HCl, 154 mM NaCl, pH 7.5). Biotinylated rabbit-anti-cystatin S polyclonal antibody (valence: 1:1000) is applied in the holes and incubated for an hour (37° C.). Wash the holes with TBS buffer and add streptavidin-peroxidase conjugate (ABC complex). Incubate the plate for 1 hour under 37° C. and wash the holes with TBS buffer. TMB solution (TMB Peroxidase Substrate, Kirkegaard and Perry Laboratories Inc. (Gaithersburg, Md.) Cat.No. 50-76-01) is added and OD at 405 nm is quantified by a microplate reader.

### Example 1

### 1. Cystatin S Detection in Breast Cancer Cell Line Culture Supernatant

CST4 mRNA is over expressed in breast cancer tumors as discussed above. As a secretion protein, cystatin S can be found in various body fluids and secretions. In order to establish cystatin S as a marker for breast cancer, the supernatant of HCC1973 (a breast cancer cell line) with high expression of CST4 mRNA was loaded on 15% polyacrylamide gel (Lanes 1-2, FIG. 14); control sample (healthy people serum) was loaded on the gel (Lane 3-4, FIG. 14). After the electrophoresis, the protein was transferred to a nitrocellulose membrane, which reacted with anti-cystatin S antibody and goat-anti-rabbit IgG with peroxidase labeling. TMB was applied for protein imaging. Methods described above were followed.

As shown in FIG. 14, β-actin (internal reference) is at the bottom of the gel. A band with 16 kDa protein was observed in Lanes 1-2 while for Lanes 3-4, the bands were very faint.

### 2. Cystatin S Detection in Breast Cancer Patients Serum

Protocol described in section “1” was followed. As shown in FIG. 15, 13-actin (internal reference) is at the bottom of the gel. A band with 16 kDa protein was observed in Lanes 1-2 (cancerous sample) while for Lanes 3-4 (control sample), the bands were very faint.

### 3. Serum Cystatin S Level Determination by ELISA Using Monoclonal Antibodies

Cystatin S (5 μg/mL) was applied on the ELISA plate, which was incubated overnight (4° C.). Fifty serum samples (30 from breast cancer patients and 20 from healthy people) were mixed with anti-cystatin S monoclonal antibody (valence 1:2000, dissolved in TBS with 3% BSA) and incubated overnight (4° C.). The sample mixture was applied on the pre-treated ELISA plate, which was incubated for an hour (ambient temperature). The plate was washed by TBS buffer and incubated with goat-anti-rabbit antibody (0.08 μg/mL, dissolved in TBS). The plate was subjected to reaction with p-nitrophenyl phosphate (p-NPP). Microplate reader is used for quantification.

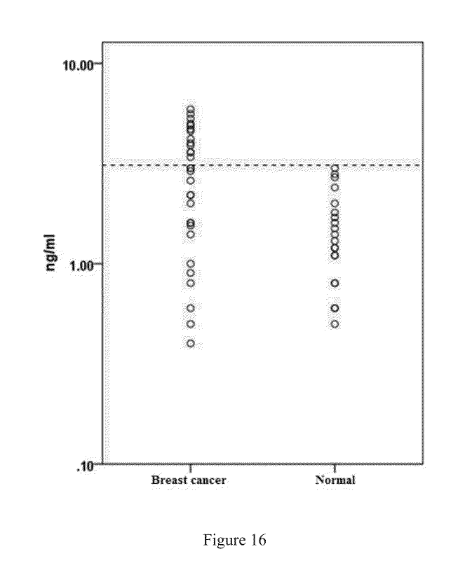

As shown in FIG. 16, the median of cystatin S level in normal serum is 1.35 ng/mL while it is 2.95 ng/mL. A cutoff vale of 3.105 ng/mL is capable for the distinguishing of cancerous samples and normal samples.

### 4. Comparisons of the Sensitivity and Specificity of Cystatin S and CEA for Breast Cancer Diagnosis and Prediction

Cystatin S was measured following the method described in section “3”. CEA was measured using commercial kit (DRG, Germany, Cat.No. EIA5071) and user manual is followed.

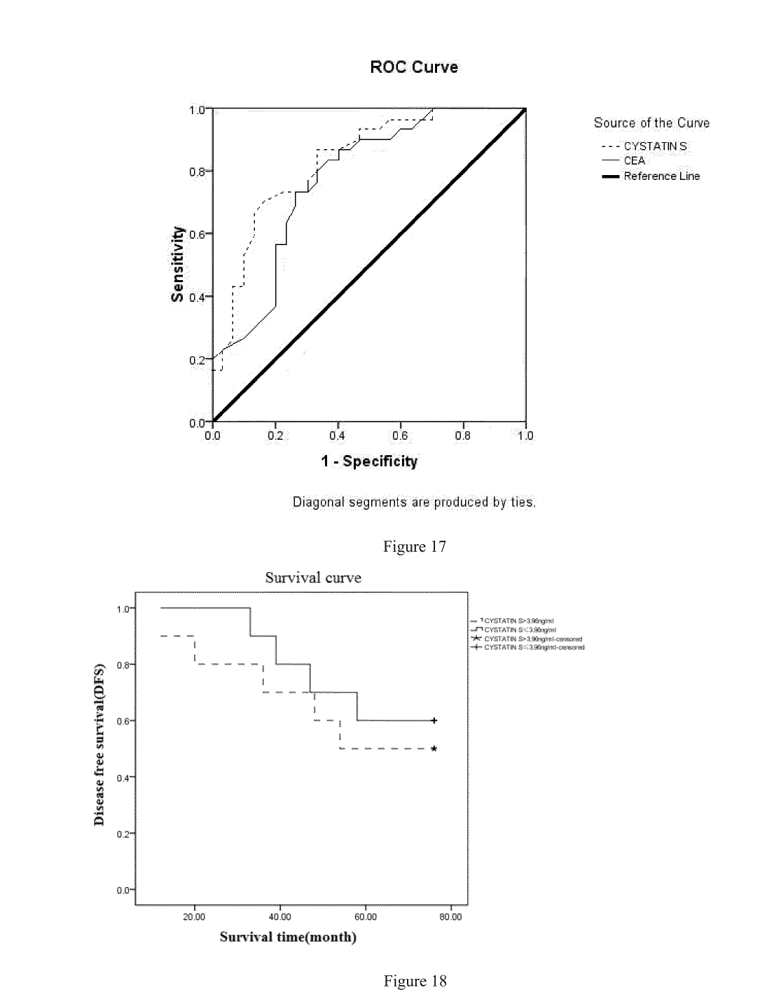

As shown in FIG. 17 and Table 3, the integration of the receiving operating characteristic (ROC) curve is 0.832 for cystatin 5 and 0.776 for CEA, which means the former has better sensitivity and selectivity.

TABLE 3
Integrations of ROC curves

| | | | P: comparison | Confidence Interval | |
|---|---|---|---|---|---|
| | Integra- | Std. | of integration | (cl 95%) | |
| Variable | tion | Dev. | and 0.5 | Lower | Caps |
| Cystatin S | 0.832 | 0.052 | 0.000 | 0.730 | 0.935 |
| CEA | 0.776 | 0.061 | 0.000 | 0.657 | 0.894 |

### 5. Methods, Testing Kits and Protocols

Methods, testing kits and protocols that are non-limiting and demonstrative.

Methods for breast diseases detection include the following Immobilize cystatin S antigen on a substrate. Apply R&D antibody (MAB 1296) to the pre-treated substrate. Wash the substrate and apply a secondary antibody such as ALP labeled goat-anti-rabbit IgG (Beyotime Inc., Cat. No. A239). Wash the substrate and measure and/or detect the amount of the protein from response from the label (ALP in the case) directly or indirectly.

The substrate include but is not limited to resin particles, cellulose-based materials such as cellulose sheets, plastic plates and particles, etc.

Antigens can be immobilized covalently or non-covalently. Sample for testing is human serum. Substrate selected or preferred should be backfilled by BSA before the addition of the sample to minimize the non-specific interaction between other components in serum and the substrate. The substate is then washed by proper buffer such as phosphate with surfactant.

A non-limiting example of the labeled secondary antibody is labeled anti-mouse polyclonal antibody. The label include and is not limited to enzymes such as ALP, luciferase, peroxidase, β-galactosidase and fluorescent dyes such as fluorocein. Molecules such as biotin, avidin, streptavidin and digitalis glycoside might be applied for the coupling of the antibody and the label molecule.

If an enzyme is the label, its qualitative and/or quantitative detection can be realized by the addition of the enzymatic substrate and the enzymatic colorimetric and/or luminescent reaction. If a fluorescent dye is the label, its qualitative and/or quantitative detection can be realized by UV exposure and fluorescence measurement/detection. Sensitizer is used if necessary. As applications of this invention, supplies that bind cystatin S or its epitopes (SEQ ID No.50) are anti-cystatin S antibodies or their fragments, secondary antibodies and solid substrates if necessary, and one or several supporting supplies. These required or optional reagents are provided in the testing kit. The reagents mentioned above might be used for breast cancer diagnosis, pTNM stage determination, metastasis detection and evaluation of the therapy effect.

For example, the testing kit features specific antibody or its fragment. It also features the reporting unit for the detections of the target protein in the sample optionally or preferably. The reporting unit is preferably a proper secondary antibody, and the label for detection optionally or preferably (if necessary). The kit optionally or preferably includes one or more buffers such as buffers for protein-substrate incubation and protein backfilling for the removal of the non-specificity interactions of proteins in the sample and proteins immobilized on the substrate, as well as washing buffers for the substrate after incubations with sample, secondary antibody and/or reagents mentioned above. Optionally, the kit provides solid substrate for control protein immobilization for competitive method. In this case, the antibody and the sample are pre-incubated and the mixture is then applied on the substrate. The reporting unit is used for the detection of the antibody and the substrate. Professionals might determine whether the antibody binds the epitope from the serum sample and quantitatively measure the amount of the antibody that binds the serum epitope. Thus the target protein amount can be determined.

Optionally, the kit can be applied in double-antibody sandwich method, wherein anti-cystatin S antibody is immobilized on the substrate. Cystatin S standard solution and pre-treated sample serum are applied on the substrate. Anti-cystatin S polyclonal antibody with reporting-unit-labeling is applied on the substrate. Professionals might determine whether the antibody binds the epitope from the serum sample and quantitatively measure the amount of the antibody that binds the serum epitope. Thus the target protein amount can be determined.

The selection of the testing reagents and/or testing kits, and/or instruments for the measurements and/or requirements for the combinations of the kit and instrument, are dependent upon the methods used for the detection. As mentioned above, these methods include and are not limited to ELISA, protein blotting and flow cytometry. ELISA method are mentioned above and blotting is more accurate but requires more equipment and/or operation time.

### 6. A Demonstrative Testing Kit and its Protocol

Sample collection and storage. For serum, the blood sample is kept for 2 hours under ambient temperature or overnight under 4° C. The sample is centrifuged for 20 minutes (1000×g) and the supernatant is collected as serum. The serum sample should be kept under −20° C. or −80° C. and repeated thawing-freezing should be avoided. For plasma, use EDTA or heparin as the anti-coagulant. Centrifuge the sample for 15 minutes (2-8° C., 1000×g) in less than 30 minutes after blood acquisition. The plasma sample should be kept under −20° C. or −80° C. and repeated thawing-freezing should be avoided. Sample pre-treatment. Serum or plasma samples are recommended to be diluted by 10 times. For instance, mix 100 μL serum or plasma sample with 900 μL PBS buffer. The samples should be diluted by 0.1 M PBS buffer (pH 7.0-7.2)

The testing kit should include the following: 1) ELISA plate closed by a plastic foil; 2) cystatin S standard solutions. Cystatin S solution is prepared with a concentration of 10 ng/mL using PBS buffer with 1% BSA. A series of solutions with concentrations of 5 ng/mL, 2.5 ng/mL, 1 ng/mL and 0.5 ng/mL are prepared by diluting the stock solution. PBS buffer with 1% BSA is used as the solution with 0 ng/mL cystatin S. The solutions should be prepared no more than 15 minutes before the testing. For example, the preparation of 4 ng/mL cystatin S can be realized by mixing 0.5 mL (no less than 0.5 mL) cystatin S solution (8 ng/mL) and 0.5 mL dilution buffer in an Eppendorf tube. Other concentrations can be realized in a similar way. 3) PBST buffer with 3% BSA for backfilling. 4) Antibody for coating: 5 μg/mL rat-anti-cystatin S monoclonal antibody and buffer for dilution (0.05 M NaHCO₃ solution, pH 9.0). 5) Biotinylated rabbit-anti-cystatin S (valence 1:200) and dilution buffer (PBST with 1% BSA). 6) ABC (streptavidin-biotin-peroxidase conjugate). 7) TMB solution for colorimetric detection. 8) PBST buffer (0.05% Tween-20 in PBS). 9) Stop solution: 2 N H₂SO₄.

### Detailed Operation Protocol is Described as Following.

1) Coating. The rat-anti-cystatin S solution is diluted to 5 μg/mL using sodium bicarbonate buffer (0.05 M, pH 9.0). The solution is applied to the holes (0.1 mL per hole) on a polystyrene plate. The plate is incubated overnight (4° C.). Discard the solution in the holes and wash the holes by washing buffer for three times with three minutes each time.

2) Backfilling. Add 200 μL PBST buffer with 3% BSA in the holes. Incubate the plate for an hour (37° C.) or overnight (4° C.). Discard the solution in the holes and wash the holes by washing buffer for three times with three minutes each time.

3) Sample loading. Assign holes for blank, standard solution and sample solution. Add 100 μL dilution buffer, standard solutions and sample solution to the blank holes, standard holes and sample holes respectively. Avoid bubbles. The solutions should be loaded at the bottom of the holes. Solution contact with hole wall should be avoided. Shake the plate gently and close the holes by a lid or plastic foil. Incubate the plate for 120 minutes (37° C.). Standard solutions should be freshly prepared to ensure the accuracy of the result.

4) Discard the remaining solution in the holes and dry the plate. Do not wash the plate. Add biotinylated rabbit-anti-cystatin S polyclonal antibody solution (200× diluted by PBST with 1% BSA). Close the holes by a lid or plastic foil. Incubate the plate for 60 minutes (37° C.).

5) Incubate the plate for 60 minutes and discard the remaining liquid in the holes. Dry the plate followed by plate washing for 3 times with 1-2 minutes soaking of 300 μL washing buffer each time. Dry the plate or shake the plate to remove the remaining liquid in the holes.

6) Add 100 μL ABC solution to the holes. Close the holes with a foil and incubate the plate for 60 minutes (37° C.).

7) Incubate the plate for 60 minutes, discard the liquid in the holes and dry the plate. Wash the plate for 5 times with 1-2 minutes soaking of 300 μL washing buffer each time. Dry the plate or shake the plate to remove the remaining liquid in the holes.

8) Successively add 50 μL enzymatic substrate solution in the holes. Close the plate with a foil and incubate the plate in darkness (37° C.) in less than 15 minutes when solution in the standard holes has blue color correlated to standard solutions concentrations and no color for the blank holes.

9) Stop the reactions by add 50 μL stop solution. The solution turns from blue to yellow. The order for stop solution addition should be identical to that of the enzymatic substrate addition. Stop solution should be immediately added when the reaction is about to the end to ensure the accuracy of the testing.

10) Read the OD value of the solutions in holes (405 nm), which occurs immediately after the stopping of the enzymatic reactions.

Calculation: OD values versus concentrations of the respective solutions are plotted. The calibration equation is acquired through data fitting and R² is calculated, which should be higher than 0.95 for an effective testing. The analyte concentration is calculated by the OD vale of the sample and the calibration equation.

### 7. Cystatin S expression for breast cancer pTNM stage determination

Experimental details are identical to section 7. Samples from 80 cytologically diagnosed breast cancer patients (20 T-stage cases, 30 N-stage cases and 30 M-stage cases). As summarized in Table 4, with the development of cancer, cystatin S expression increases, which indicates that cystatin S protein expression can be used for pTNM stage determination.

TABLE 4
Cystatin S expression of breast cancer
patients with various pTNM stages

| | Median of cystatin S expression (ng/mL) |
|---|---|
| T-stage (20 cases) | 1.16 |
| N-stage (30 cases) | 2.89 |
| M-stage (30 cases) | 4.34 |

### 8. Cystatin S Expression for Breast Cancer Metastasis Diagnosis

Experimental details are identical to section 7. Samples from 50 cytologically diagnosed breast cancer patients, among which 30 patients have cancer metastasis. As summarized in Table 5, cystatin S expression is higher in those with metastasis than those without cancerous metastasis, indicating that cystatin S expression is a marker for breast cancer metastasis diagnosis.

TABLE 5
Cystatin S expression of breast cancer
patients with and without metastasis

| | Median of cystatin S expression (ng/mL) |
|---|---|
| No metastasis (20 cases) | 1.43 |
| Metastatic cancer (30 cases) | 4.19 |

### 9. Cystatin S Expression for the Evaluation of the Endocrine Therapy Combined with Chemotherapy for the Treatment of Breast Cancer

Experimental details are identical to section 7. 2885 breast cancer (N0-1 stages) were studied. The patients were treated with four cycles of AC (doxorubicin+cyclophosphamide) or AT (doxorubicin+paclitaxel). Endocrine therapy was prosecuted based on the hormone receptor condition. All patients were followed up for 76 months. The cystatin S expression was measured after the last cycle of the therapy. 776 cases were validated in the study, whose cystatin S expression median was 3.96 ng/mL. As shown in FIG. 19, disease-free survival (DFS) for patients with higher cystatin S expression than the median was 49%, lower than that of the group with lower cystatin S expression than the median, which was 64%.

It should be noted that all examples describe some feature of the invention for better and clearer presentation. The can be combined in one practice. All features or sub-features can be combined if necessary.

Methods in this invention were illustrated and presented by examples. Many substitutions, modifications and changes are straight forward to professional in this field. Any efforts of these substitutions, modifications and changes are part of the claims and their reasonable extensions. Any publications, patents and patent applications referenced in this patent should be used to illustrate the details for experimental procedures and protocols that are not included in this invention. Any references in mentioned in this invention are not acknowledged as a substitute of the technologies of this invention.

1. The applications of the following in the predictions and diagnoses of breast cancers are disclosed in this invention: CST4 gene, mRNA of CST4, cDNA of the splices of CST4, cystatin S (a protein coded by CST4) and the epitope of cystatin S. The sequence of cystatin S is shown in SEQ ID No.42.

2. A method according to claim 1, wherein the probe sequences of CST4, mRNA of CST4 and cDNA of the splices of CST4 are shown in SEQ ID No.3.

3. A method according to claim 1, wherein the specific primers of the amplicon have sequences shown in SEQ ID No.1, 4, 6, 8, 10, 12, 14, 16, 18, 20 (primer 1) and in SEQ ID No.2, 5, 7, 9, 11, 13, 15, 17, 19, 21 (primer 2). Sequence in SEQ ID No.1 pairs with sequence in SEQ ID No.2. Sequence in SEQ ID No.4 pairs with sequence in SEQ ID No.5. Sequence in SEQ ID No.6 pairs with sequence in SEQ ID No.7. Sequence in SEQ ID No.8 pairs with sequence in SEQ ID No.9. Sequence in SEQ ID No.10 pairs with sequence in SEQ ID No.11. Sequence in SEQ ID No.12 pairs with sequence in SEQ ID No.13. Sequence in SEQ ID No.14 pairs with sequence in SEQ ID No.15. Sequence in SEQ ID No.16 pairs with sequence in SEQ ID No.17. Sequence in SEQ ID No.18 pairs with sequence in SEQ ID No.19. Sequence in SEQ ID No.20 pairs with sequence in SEQ ID No.21.

4. A method according to claim 1, wherein the sequence of the epitope peptide of cystatin S is shown in SEQ ID No.50.

5. A method according to claim 1, wherein the applications include the metastasis, micro-metastasis, pTNM staging of breast cancers, real time monitoring of the tumor during cancer treatment and prognosis predictions.

6. Capturers for the biomarkers of breast cancers, wherein the capturers are for markers for breast cancer prediction and diagnosis. Markers for the breast cancer are CST4 gene, mRNA of CST4, cDNA of the splices of CST4, amplicons of CST4-specific primers, cystatin S coded by CST4 and epitope peptide of cystatin S.

7. Capturers according to claim 6, wherein sequences for the primers for CST4 are shown in SEQ ID No.1-2.

8. Capturers according to claim 6, wherein the sequence for the probe for CST4, mRNA of CST4 or splices of the latter is shown in SEQ ID No.3.

9. Capturers according to claim 6, wherein the sequence for the amplicon is shown in SEQ ID No.43.

10. Capturers according to claim 6, wherein capturers for cystatin S include specific antibodies for cystatin S or its epitope peptides.

11. Capturers according to claim 6, wherein the sequence of epitope peptide of cystatin S is presented in SEQ ID No.50.

12. Applications of these capturers mentioned above in the manufacturing of testing reagents and kits for breast cancer detections.

13. Testing kits according to claim 6, wherein the capturers are included.

14. Testing kits according to claim 13, wherein the detailed descriptions are as following:
1) Real time and quantitative testing kits for mRNA of CST4 using TaqMan probes. The primers sequences are shown in SEQ ID No. 1-2. The sequence of the probe is shown in SEQ ID No.3.
2) Real time and quantitative testing kits for mRNA of CST4 using fluorescent dyes as probes. The primers sequences are shown in SEQ ID No. 1-2. The sequences of the primers for internal reference are shown in SEQ ID No.30-31. Or
3) Quantitative testing kits for mRNA of CST4 based on nucleic acid based amplification (NASBA) or transcription-median amplification (TMA). Both kits include primers and probes for CST4, whose sequences are shown in SEQ ID No.2, 32 (for primers) and 3 (for probe). Or
4) Quantitative testing kits for mRNA of CST4 based on ligase chain reaction (LCR). Four probes are included whose sequences are shown in SEQ ID No.33-36. Or
5) Quantitative testing kits for mRNA of CST4 based on thermophilic strand displacement amplification (tSDA). Primers (sequences shown in SEQ ID No.37-40) and a probe (SEQ ID No. 41) are included.

15. Testing kits according to claim 13, wherein the detailed descriptions are as following:
1) Double-antibody sandwich ELISA kits, including the solid substrate, capturers immobilized on the solid substrate, biotinylated capturers and enzymatic substrate (colorimetric). Capturers immobilized are monoclonal antibodies while biotinylated capturers are polyclonal antibodies. Or
2) Blotting kits including solid substrate, capturers, enzymatic labeled secondary antibody and enzymatic substrate for colorimetric detections. The capturers are monoclonal antibodies and biotinylated capturers are polyclonal antibodies. Or
3) Competitive ELISA kits including solid substrate, immobilized antigen, biotinylated capturers, the enzymatic substrate for colorimetric detections and specific monoclonal antibody. The biotinylated capturers are polyclonal antibodies.

16. Testing kits according to claim 14, wherein positive and negative controls and blank samples are included.

17. Double-antibody ELISA testing kits according to claim 15, wherein the monoclonal antibody is rat-anti-cystatin S antibody; the solid substrate is ELISA plate and the biotinylated polyclonal antibody is biotinylated rabbit-anti-cystatin S polyclonal antibody.

18. Protocols of the testing kits according to claim 17, wherein the details are described as following: Coat the ELISA plate by rat-anti-cystatin S antibody, which is backfilled by 3% BSA afterwards. Apply samples with eight-fold dilution to the plate and incubate it under 37° C. Wash the holes with samples by TBS and add biotinylated rabbit-anti-cystatin S polyclonal antibody. Incubate the plate under 37° C. Wash the holes with samples by TBS and add streptavidin-biotin-horseraddish peroxidase (HRP) complex. Incubate the plate under 37° C., followed by plate washing by TBS. Finally, the analyte is quantified by the addition of alkaline phosphatase (ALP) and reading of QD (405 nm) on a microplate reader.

19. A method according to claim 14 using breast cancer diagnosis testing kits or for breast cancer predictions, wherein the expression level or the quantitative content of the breast cancer markers measured via the testing kits is compared with those of healthy people to decide whether the result is positive or not; or the result is read positive if it is higher than a cutoff value. The cutoff value is obtained through the comparison of the breast cancer markers expressions/levels in the body fluids or tissue samples of breast cancer patients and healthy people. The cutoff value is of statistical significance. Samples include one or more of the following: blood, urine, marrow, breast cancer cell lines, breast cancer tumors and tumor adjacent tissues and lymph node tissues.

20. Testing kits for the prediction and diagnosis of breast cancer, wherein the kits detect blood cystatin S level and wherein the kit includes solid substrate, capturers immobilized on the substrate and biotinylated capturers and the corresponding substrate for colorimetric detection. Immobilized captueres are specific monoclonal antibodies; capturers that are biotinylated are polyclonal antibodies.
Or the kits detect blood cystatin S level, which include solid substrate, immobilized cystatin S on the substrate, mouse-anti-cystatin S monoclonal antibody, enzymatic labeled secondary antibody and the corresponding substrate for colorimetric detection.
Or the kits detect blood cystatin S level, which include solid substrate, capturers, enzymatic labeled secondary antibody and, the corresponding substrate for colorimetric detection. Capturers include specific monoclonal antibodies; capturers that are biotinylated are polyclonal antibodies.

21. A testing kit according to claim 15 based on double-antibody sandwich ELISA, wherein the ELISA plate is the solid substrate, wherein the immobilized capturer is rat-anti-cystatin S monoclonal antibody, wherein the biotinylated capturer is rabbit-anti-cystatin S polyclonal antibody (with valence of 1:1000) and wherein the substrate for colorimetric deletion is alkaline phosphatase.
Or the kit is based on competitive ELISA, wherein the ELISA plate is the solid substrate, wherein the concentration of cystatin S is 5 μg/mL, wherein the specific monoclonal antibody is rat-anti-cystatin S antibody (with valence of 1:2000), wherein enzymatic labeled secondary antibody is ALP-labeled goat-anti-mouse IgG (with valence of 1:2000) and wherein the substrate for colorimetric detetion is ALP substrate. The volume ratio of cystatin S, enzymatic labeled secondary antibody and ALP substrate is 1:2.
Or the kit is based on immunoblotting, wherein the solid substrate is nitrocellulose membrane, wherein the capturer is monoclonal cystatin S antibody (with valence of 1:1000), wherein the enzymatic labeled secondary antibody is peroxidase labeled goat-anti-rabbit IgG and wherein the enzymatic substrate is TMB solution.


SEQUENCE LISTING (50 sequences)

SEQ ID NO: 1 - DNA, 20 bp, Artificial Sequence
  Oligonucleotide
gctctcaccctcctctcctg

SEQ ID NO: 2 - DNA, 20 bp, Artificial Sequence
  Oligonucleotide
tatcctattctcctccttgg

SEQ ID NO: 3 - DNA, 24 bp, Artificial Sequence
  Oligonucleotide
ctccagctttgtgctctgcctctg

SEQ ID NO: 4 - DNA, 23 bp, Artificial Sequence
  Oligonucleotide
agtacaacaaggccaccgaagat

SEQ ID NO: 5 - DNA, 23 bp, Artificial Sequence
  Oligonucleotide
agaagcaagaaggaaggagggag

SEQ ID NO: 6 - DNA, 23 bp, Artificial Sequence
  Oligonucleotide
tacaacaaggccaccgaagatga

SEQ ID NO: 7 - DNA, 23 bp, Artificial Sequence
  Oligonucleotide
agaagcaagaaggaaggagggag

SEQ ID NO: 8 - DNA, 23 bp, Artificial Sequence
  Oligonucleotide
tgctactcctgatggctaccctg

SEQ ID NO: 9 - DNA, 23 bp, Artificial Sequence
  Oligonucleotide
gtggccttgttgtactcgctgat

SEQ ID NO: 10 - DNA, 23 bp, Artificial Sequence
  Oligonucleotide
agtacaacaaggccaccgaagat

SEQ ID NO: 11 - DNA, 27 bp, Artificial Sequence
  Oligonucleotide
taccaggtctattagaagcaagaagga

SEQ ID NO: 12 - DNA, 23 bp, Artificial Sequence
  Oligonucleotide
tgctactcctgatggctaccctg

SEQ ID NO: 13 - DNA, 23 bp, Artificial Sequence
  Oligonucleotide
catcttcggtggccttgttgtac

SEQ ID NO: 14 - DNA, 23 bp, Artificial Sequence
  Oligonucleotide
tgctactcctgatggctaccctg

SEQ ID NO: 15 - DNA, 23 bp, Artificial Sequence
  Oligonucleotide
tactcatcttcggtggccttgtt

SEQ ID NO: 16 - DNA, 25 bp, Artificial Sequence
  Oligonucleotide
tgggattatcctattctcctccttg

SEQ ID NO: 17 - DNA, 23 bp, Artificial Sequence
  Oligonucleotide
ctccagctttgtgctctgcctct

SEQ ID NO: 18 - DNA, 23 bp, Artificial Sequence
  Oligonucleotide
tgctactcctgatggctaccctg

SEQ ID NO: 19 - DNA, 23 bp, Artificial Sequence
  Oligonucleotide
ctcatcttcggtggccttgttgt

SEQ ID NO: 20 - DNA, 23 bp, Artificial Sequence
  Oligonucleotide
tacagtgggtgggagtgggtggt

SEQ ID NO: 21 - DNA, 23 bp, Artificial Sequence
  Oligonucleotide
gagtgggtacagcgtgcccttca

SEQ ID NO: 22 - DNA, 24 bp, Artificial Sequence
  Oligonucleotide
tgcctcgggctctcaccctcctct

SEQ ID NO: 23 - DNA, 24 bp, Artificial Sequence
  Oligonucleotide
tgggtggtggtcggtgtgactggc

SEQ ID NO: 24 - DNA, 19 bp, Artificial Sequence
  Oligonucleotide
agtcccagcccaacttgga

SEQ ID NO: 25 - DNA, 24 bp, Artificial Sequence
  Oligonucleotide
gggaacttcgtagatctggaaaga

SEQ ID NO: 26 - DNA, 20 bp, Artificial Sequence
  Oligonucleotide
cagaagaaacagttgtgctc

SEQ ID NO: 27 - DNA, 18 bp, Artificial Sequence
  Oligonucleotide
ggagtaggaggtggtcag

SEQ ID NO: 28 - DNA, 18 bp, Artificial Sequence
  Oligonucleotide
tctcaccctcctctcctg

SEQ ID NO: 29 - DNA, 21 bp, Artificial Sequence
  Oligonucleotide
ttatcctatcctcctccttgg

SEQ ID NO: 30 - DNA, 19 bp, Artificial Sequence
  Oligonucleotide
aagatcattgctcctcctg

SEQ ID NO: 31 - DNA, 18 bp, Artificial Sequence
  Oligonucleotide
cgtcatactcctgcttgc

SEQ ID NO: 32 - DNA, 45 bp, Artificial Sequence
  Oligonucleotide
aattctaatacgactcactataggggctctcaccctcctctcctg

SEQ ID NO: 33 - DNA, 25 bp, Artificial Sequence
  Oligonucleotide
gggctctggcctcgagctccaagga

SEQ ID NO: 34 - DNA, 30 bp, Artificial Sequence
  Oligonucleotide
ataggataatcccaggtggcatctatgatg

SEQ ID NO: 35 - DNA, 30 bp, Artificial Sequence
  Oligonucleotide
tctcctccttggagctcgaggccagagccc

SEQ ID NO: 36 - DNA, 30 bp, Artificial Sequence
  Oligonucleotide
catcatagatgccacctgggattatcctat

SEQ ID NO: 37 - DNA, 23 bp, Artificial Sequence
  Oligonucleotide
cccggcctctgtgtaccctgcta

SEQ ID NO: 38 - DNA, 30 bp, Artificial Sequence
  Oligonucleotide
gaactcgagctaccctggctggggctctgg

SEQ ID NO: 39 - DNA, 24 bp, Artificial Sequence
  Oligonucleotide
ggtggccttgttgtactcgctgat

SEQ ID NO: 40 - DNA, 28 bp, Artificial Sequence
  Oligonucleotide
gctctcgagagtgaagggcacgctgtac

SEQ ID NO: 41 - DNA, 29 bp, Artificial Sequence
  Oligonucleotide
ttactcgagctccaaggaggagaatagga

SEQ ID NO: 42 - DNA, 736 bp, Homo sapiens
ggctctcaccctcctctcctgcagctccagctttgtgctctgcctctgaggagaccatggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggaggacagaatgtccctggtgaattccaggtgtcaagaagcctaggggtctgtgccaggccagtcacaccgaccaccacccactcccacccactgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccccacctcctgcaattaaacagtagcatcgcc

SEQ ID NO: 43 - DNA, 142 bp, Homo sapiens
gctctcaccctcctctcctgcagctccagctttgtgctctgcctctgaggagaccatggcccggcctctgtgtaccctgctactcctgatggctaccctggctggggctctggcctcgagctccaaggaggagaataggata

SEQ ID NO: 44 - DNA, 759 bp, Homo sapiens
gggctccctgcctcgggctctcaccctcctctcctgcagctccagctttgtgctctgcctctgaggagaccatggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggaggacagaatgtccctggtgaattccaggtgtcaagaagcctaggggtctgtgccaggccagtcacaccgaccaccacccactcccacccactgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccccacctcctgcaattaaacagtagcatcgcctccctctg

SEQ ID NO: 45 - DNA, 284 bp, Homo sapiens
ggctctcaccctcctctcctgcagctccagctttgtgctctgcctctgaggagaccatggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcag

SEQ ID NO: 46 - DNA, 114 bp, Homo sapiens
acctttgggggggtgaattacttcttcgacgtagaggtgggccgcaccatatgtaccaagtcccagcccaacttggacacctgtgccttccatgaacagccagaactgcagaag

SEQ ID NO: 47 - DNA, 338 bp, Homo sapiens
aaacagttgtgctctttcgagatctacgaagttccctgggaggacagaatgtccctggtgaattccaggtgtcaagaagcctaggggtctgtgccaggccagtcacaccgaccaccacccactcccacccactgtagtgctcccacccctggactggtggcccccaccctgcgggaggcctccccatgtgcctgtgccaagagacagacagagaaggctgcaggagtcctttgttgctcagcagggcgctctgccctccctccttccttcttgcttctaatagacctggtacatggtacacacacccccacctcctgcaattaaacagtagcatcgcc

SEQ ID NO: 48 - protein, 141 aa, Homo sapiens
MARPLCTLLLLMATLAGALASSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA

SEQ ID NO: 49 - DNA, 426 bp, Homo sapiens
atggcccggcctctgtgtaccctgctactcctgatggctaccctggctggggctctggcctcgagctccaaggaggagaataggataatcccaggtggcatctatgatgcagacctcaatgatgagtgggtacagcgtgcccttcacttcgccatcagcgagtacaacaaggccaccgaagatgagtactacagacgcccgctgcaggtgctgcgagccagggagcagacctttgggggggtgaattacttcttcgacgtagaggtgggccgcaccatatgtaccaagtcccagcccaacttggacacctgtgccttccatgaacagccagaactgcagaagaaacagttgtgctctttcgagatctacgaagttccctgggaggacagaatgtccctggtgaattccaggtgtcaagaagcctag

SEQ ID NO: 50 - protein, 121 aa, Homo sapiens
SSSKEENRIIPGGIYDADLNDEWVQRALHFAISEYNKATEDEYYRRPLQVLRAREQTFGGVNYFFDVEVGRTICTKSQPNLDTCAFHEQPELQKKQLCSFEIYEVPWEDRMSLVNSRCQEA